(19) United States
(12) Patent Application Publication
(10) Pub. No.: US 2022/0160647 A1
(43) Pub. Date: May 26, 2022

(54) TRANSDERMAL DRUG DELIVERY DEVICE/TRANSDERMAL PATCH AND OTHER APPLICATIONS OF EMULSION FORMULATION

(71) Applicant: First Necessity, LLC, Stow, OH (US)

(72) Inventors: Anthony S. Giovengo, Kent, OH (US); Brandon S. True, Parma, OH (US)

(73) Assignee: First Necessity, LLC, Stow, OH (US)

(21) Appl. No.: 17/534,801

(22) Filed: Nov. 24, 2021

Related U.S. Application Data
(60) Provisional application No. 63/117,531, filed on Nov. 24, 2020.

(51) Int. Cl. A61K 9/70; A61K 47/36; A61K 47/24; A61K 47/10; A61K 31/05; A61K 31/714; A61K 9/127; A61K 31/352; A61K 47/32; A61K 47/14

(52) U.S. Cl. CPC A61K 9/7084; A61K 47/36; A61K 47/24; A61K 47/10; A61K 47/14; A61K 31/714; A61K 9/127; A61K 31/352; A61K 47/32; A61K 31/05

(57) ABSTRACT

The present disclosure relates to a transdermal drug delivery device, also known as a transdermal patch that can be worn by the user anywhere the person travels throughout the day. The transdermal patch is characterized in that it is a single-layered patch comprising an liposomal emulsion carrying a drug to be administered and an adhesive which are both incorporated into a polymer or polymeric matrix. The liposomal emulsion within the single-layered transdermal patch allows the drug that is to be administered to be spread throughout the entire surface area of the transdermal patch.

### TECHNICAL FIELD

The present disclosure relates to a transdermal patch which incorporates an emulsion formulation that can be worn by the user for administering or delivering drugs into the body, in particular cannabis. The present disclosure also relates to other applications of an emulsion formulation described in greater detail below that may be worn, applied or ingested by the user to administer or deliver drugs such as cannabis into the body.

### BACKGROUND

The use of transdermal patches for delivering drugs through the skin is well known. However, transdermal drug delivery devices known in the art have a number of drawbacks. First, the transdermal drug delivery device typically contains a drug reservoir that only covers a portion of the patch leaving a large amount of surface area unused for drug delivery. The reason for this is that a portion of the surface area must be used for holding an adhesive layer to adhere the patch to the user's skin. This presents an inherent inefficiency in drug delivery into the skin of the user in that the full potential of drug delivery is not utilized.

In addition, transdermal drug delivery devices known in the art typically contain a number of layers. These layers include a backing layer, a drug reservoir, an adhesive layer, a rate controlling membrane and a release liner. These multiple layers introduce a level of complexity into the device that results in not only increased costs but inefficient and less effective drug delivery.

Also many transdermal drug delivery devices in the art have a color which is similar to that of various skin tones. This makes the patch readily visible and apparent on the user wearing it and presents a disadvantage to users who wish to have a more discreet manner of wearing a patch for without drawing attention to themselves, particularly with respect to the administration of cannabis-type drugs.

What is needed in the art is a simpler transdermal drug delivery device that is transparent, that does not require a multiple layered device as in the prior art and that is capable of more efficiently and more effectively delivering drugs into the user's bloodstream. This and other types of products are discussed in detail below.

### SUMMARY

Provided is a transdermal drug delivery device comprising a single-layered polymeric matrix including an emulsion having a core drug reservoir and an adhesive.

According to further aspects of the present teaching, the emulsion is a liposomal emulsion.

According to further aspects of the present teaching, the liposomal emulsion is a nanoparticle liposomal emulsion.

According to further aspects of the present teaching, the nanoparticle liposomal emulsion holds oil soluble compounds within its core.

According to further aspects of the present teaching, the oil soluble compounds within the core comprise at least one of cannabidiol (CBD), cannibigerol (CBG) and tetrahydrocannabinol (THC).

According to further aspects of the present teaching, the polymeric matrix comprises an acrylates copolymer.

According to further aspects of the present teaching, the adhesive comprises one or more of the following solvents: ethyl acetate, heptanes, n-heptane, hexane, methanol, ethanol, isopropanol, 2,4-pentanedione, toluene, and xylene.

According to further aspects of the present teaching, the adhesive comprises ethyl acetate.

According to further aspects of the present teaching, the transdermal drug delivery device has a transparency of greater than 80%.

According to further aspects of the present teaching, the emulsion further includes hyaluronic acid.

According to further aspects of the present teaching, the polymeric matrix further includes vitamin B12 and natural carrier proteins outside of the liposomes within the polymeric matrix.

According to further aspects of the present teaching, the onset time is from about 5 to about 15 minutes.

According to further aspects of the present teaching, a substantial portion of the drug (for example, about 80% of the drug) is administered in about 30 to about 60 minutes.

According to further aspects of the present teaching, the core includes vitamin B12.

According to further aspects of the present teaching, indicia is printed on a non-delivering side within the polymeric matrix.

According to further aspects of the present teaching, the transdermal drug delivery device includes a patch and further includes one of the following formulations:
i) 39.35 to 39.55 grams of emulsion and 239.05 to 239.25 grams of adhesive for producing patches measuring about 1.75 inches by about 1.75 inches that contains about 12 mg of CBD, wherein the emulsion is prepared from 10.3 to 10.5 grams of broad spectrum CBD extract, 18.9 to 19.1 grams medium chain triglyceride oil, 5.4 to 5.6 grams of water, 3.4 to 3.6 grams of ethanol, 0.9 to 1.1 grams of lecithin, and 0 to 0.15 grams of hyaluronic acid;
ii) 39.35 to 39.55 grams of emulsion and 239.05 to 239.25 grams of adhesive for producing patches measuring about 1.75 inches by about 1.75 inches that contain about 12 mg of cannabinoids in a 1:1 ratio of CBD to CBG, wherein the emulsion is prepared from 5.1 to 5.3 grams of broad spectrum CBD extract, 5.1 to 5.3 grams of CBG distillate, 18.9 to 19.1 grams medium chain triglyceride oil, 5.4 to 5.6 grams of water, 3.4 to 3.6 grams of ethanol, 0.9 to 1.1 grams of lecithin, and 0 to 0.15 grams of hyaluronic acid;
iii.) 39.35 to 39.55 grams of emulsion and 239.05 to 239.25 grams of adhesive for producing patches measuring about 1.75 inches by about 1.75 inches that contain about 12 mg of cannabinoids in a 7:1 ratio of CBG to CBD, wherein the emulsion is prepared from 1.2 to 1.4 grams of broad spectrum CBD extract, 9.0 to 9.2 grams of CBG distillate, 18.9 to 19.1 grams medium chain triglyceride oil, 5.4 to 5.6 grams of water, 3.4 to 3.6 grams of ethanol, 0.9 to 1.1 grams of lecithin, and 0 to 0.15 grams of hyaluronic acid.

Also provided is a gum comprising a polymeric matrix comprising an emulsion having a core drug reservoir and an adhesive.

According to further aspects of the present teaching, the emulsion within the gum is a liposomal emulsion.

According to further aspects of the present teaching, the liposomal emulsion within the gum is a nanoparticle liposomal emulsion.

According to further aspects of the present teaching, the nanoparticle liposomal emulsion within the gum holds oil soluble compounds within its core.

According to further aspects of the present teaching, the oil soluble compounds within the core of the liposomal emulsion within the gum include at least one of cannabidiol (CBD) and tetrahydrocannabinol (THC).

### DETAILED DESCRIPTION

The present disclosure, as demonstrated by the exemplary embodiments disclosed herein, provides a transdermal drug delivery device or patch that can be worn by a user for the delivery of drugs into the bloodstream. The transdermal drug delivery device is useful for the administration of medical cannabinoids as well as other types of drugs for treatment of various medical conditions. The transdermal drug delivery device incorporates the use of a unique emulsion formulation. This formulation may also be used in many other applications including medical devices, edibles, solutions and numerous other products as discussed in greater detail below.

The transdermal drug delivery device of the present disclosure is characterized in that it utilizes liposomal retention for carrying the drugs that are to be administered. For example, according to certain aspects of the present disclosure, drugs such as cannabidiol (CBD) and tetrahydrocannabinol (THC) are suspended in liposomes. These liposomes include a lipid bilayer and an aqueous core which are designed to hold oil soluble compounds such as CBD and THC.

According to certain aspects of the present teaching, it is desirable that the liposomes utilized in the transdermal drug delivery device be in the form of liposomal nanoparticles. This allows for the drugs such as CBD and THC that are to be delivered to be suspended within liposomal nanoparticles. The liposomes are designed to hold oil soluble compounds inside within the core and have a water soluble exterior which mimics that of a cellular membrane. The water soluble exterior or hydrophilic layer and hydrophobic layer of the liposomes allows for the liposomes to be absorbed by cells once they come in contact with the cellular membrane. This allows for the liposomes to release its contents, i.e., the drugs stored within its core within the interior of the cell. It is noted that the liposomes within the transdermal drug delivery device function as both a drug delivery system and as an emulsifier within a polymeric matrix.

The transdermal drug delivery device of the present disclosure is a single layered patch comprising an emulsion which is incorporated or integrated within a polymer or polymeric matrix. According to certain aspects of the present teaching, the polymeric matrix comprises an acrylates copolymer although other types of polymers are also contemplated for forming the polymeric matrix. The transdermal drug delivery device also includes also includes an adhesive which is incorporated into and part of the single layered polymeric matrix. In certain embodiments, the adhesive may include one or more of the following as solvents: ethyl acetate, heptanes, n-heptane, hexane, methanol, ethanol, isopropanol, 2, 4-pentanedione, toluene, and xylene.

Use of liposomes as part of the emulsion provides a number of advantages as it increases solubility of the emulsion, increases bioavailability of the drug, increases the rate of delivery of the drug, reduces onset time of the drug, minimizes side effects from the drug and broadens the potential drugs that can be delivered within the transdermal device. Compared to pure water or pure oil as a carrier, even a simple emulsion will provide benefits in these categories. The liposomes, however, significantly increase the magnitude of these benefits to the point of making the product viable versus, for example, a dosage or absorption rate improvement too low to provide noticeable effects. For example, lipid soluble drugs are typically very difficult to deliver into the body through use of a transdermal patch. This is primarily due to the protective role the skin plays in biology and its physical properties. Skin is composed of multiple layers, each with properties ranging from very hydrophobic, or water insoluble, to very hydrophilic or water soluble. As such, successful delivery requires drugs to traverse these opposing regimes. Transdermal patches by their very design are almost exclusively hydrophobic in nature. Without modification or some carrier particle, these lipid soluble drugs would remain trapped in either the patch itself or on the surface of the skin. The liposomal emulsion imparts an amphoteric solubility onto the encapsulated drug, allowing it to dissolve in either hydrophobic or hydrophilic conditions. However, the polymeric-emulsion single layer of the transdermal drug delivery device of the present disclosure allows for delivery of such lipid soluble drugs within the body.

According to further aspects of the present teaching, the transdermal drug delivery device is an edge-to-edge clear transdermal patch having various degrees of transparency. Transparency is achieved by the combination of the liposomal emulsion and the adhesive. Moreover, transparency of the transdermal drug delivery device may be altered between various degrees through selection of the adhesive and by modifying the properties of both the liposomal emulsion and the adhesive. For example, by balancing the interaction strength of the emulsifier between the hydrophobic and hydrophilic solvents and the particle size of the liposomes, the cloudiness typically seen in oil-water mixtures may be eliminated at various degrees. In certain embodiments, the transdermal patch is translucent in that it may have a transparency of at least 50% or greater. However, it is also contemplated that the transdermal patch may be up to 80% transparent while in other embodiments the transdermal patch may have a transparency that is greater than 80%.

For example, when applied at a wet (uncured) thickness of 0.015″, a composition of 14% emulsion and 86% adhesive, a transparency of 80% or about 80% is achieved. When prepared in a ratio of 90% or greater adhesive, transparencies over 80% are achievable. A critical point exists where the final product is not able to cure properly and adhere to the skin. This occurs before a transparency of 50% is observed.

In another example, when using broad spectrum CBD extract, an emulsion comprised of 30% CBD by weight added to adhesive at 14% by weight produces a net transparency of about 80%. The majority of opacity is attributable to residual waxes from the broad-spectrum extract. Greater dosages of pure CBD distillate may be achieved at greater levels of transparency, but may result in lessened efficacy due to the loss of the entourage effect driven by the minor cannabinoids of the broad spectrum extract. An emulsion comprised of 20% broad spectrum CBD extract by weight added to adhesive at 14% by weight or a 30% CBD extract added at 10% by weight to adhesive can produce transparencies over 80%.

The transdermal drug delivery device allows for faster and more efficient delivery of drugs within the bloodstream of the user than typical patches on the market. In fact, the efficiency of the transdermal drug delivery device allows for a lower dose of the drug present in the device which in turn results in a high concentration of the drug present in the patient in a manner which allows for less side effects than traditional patches.

According to further aspects of the present teaching, the emulsion that is present in the polymeric matrix of the transdermal delivery device may include any number of additional agents. For example, in certain embodiments, the emulsion may include hyaluronic acid which promotes healthy skin and prevents any irritation of the skin that may result from application of the patch or transdermal delivery device.

According to further aspects of the present teaching, polymeric matrix liposome emulsion may further include vitamin B12 and natural carrier proteins for vitamin B12 incorporated in the polymeric matrix. The vitamin B12 forms a pi stack or weak bond within the polymeric matrix. This vitamin B12 may function as a way to transfer or direct any non-liposome-incorporated cannabinoids directly present within the polymeric matrix (i.e., any cannabinoids that did not get fully encapsulated into the liposomes) into the body. The vitamin B12 and natural carrier proteins in the polymeric matrix have a catalytic effect on drug delivery in that it effectively pulls up additional cannabinoids present within the polymeric matrix thereby increasing the bioavailability of utilization of all cannabinoids within the transdermal drug delivery device. This reaction may occur after the primary delivery of the cannabinoids from the liposomes into the body in that it would provide a slower rate delivery of cannabinoids from the polymeric matrix compared to the much faster rate of delivery of cannabinoids from the liposomes. Thus, this process contemplates a more complete delivery of all or substantially all cannabinoids from the transdermal drug delivery device or patch.

Three vitamin B12 natural carrier proteins incorporated into the polymeric matrix of the transdermal drug delivery device or patch as described in the above-referenced process include transcobalamin, haptocorrin and intrinsic factor. To understand the effect vitamin B12 natural carrier proteins have on the pulling up additional cannabinoids within the polymeric matrix of the transdermal drug delivery device, one may consider the natural processes of these proteins. For instance, haptocorrin has multiple roles, the most significant of which occurs in saliva where it binds ingested vitamin B12 while chewing and protects it from degradation in the stomach by acid and/or bile. Without this protection, cobalamins (vitamin B12) are not absorbed. Next, intrinsic factor receives cobalamins in a handoff-like event from haptocorrin in the intestines and allows passage to the blood stream through the cubulin receptor. At this point cobalamins destined for delivery to broader tissues of the body are bound by transcobalamin and are distributed to their target cells through the transcobalamin receptor. Unbound cobalamins, by and large are not absorbed and are eventually excreted by the kidneys. Haptocorrin is also present in the blood stream and shuttles some amount of the cobalamins for further processing by the liver and/or kidneys although the specifics of these mechanisms have yet to be elucidated. Taking these details into consideration one can see how the delivery mode of products dictates the level of involvement of the carrier proteins.

The above-described delivery process is broader than just cobalamins however, as data suggests that compounds featuring aromatic ring functional groups form stable, but reversible interactions with cobalamins without interfering with carrier protein binding. While the exact nature of this interaction is yet to be elucidated, it is believed to be the result of π-π stacking and is observable on the minutes time scale with dramatic changes to the UV absorption spectra of the solution. This method allows one to leverage the endogenous cobalamin processing and transport system for the delivery of products to target tissues based upon the expression of Transcobalamin Receptors.

According to further aspects of the present teaching, it is contemplated that the onset time of the transdermal drug delivery device, i.e., the time where the user begins feeling the effects of delivery of the drug into the bloodstream is from about 5 to about 15 minutes.

According to further aspects of the present teaching, it is contemplated that most or substantially all of the drug content of the transdermal drug delivery device or patch (e.g., up to about 80% or more of the drug) will be delivered in about 30 to about 60 minutes. However, keeping the patch on for a period of time after the initial hour (e.g., up to an additional hour or more) may result in additional delivery of the drug into the body although at much lower amounts. After delivery of the drug through the skin, it is contemplated that the drug will accumulate and form a reservoir in the adipose tissue under the skin. Thus, it is contemplated that the drug will remain effective for a period of time and continued to be delivered to the bloodstream of the individual after the patch is removed from the skin.

In addition to being used for transdermal delivery of cannabinoids, the transdermal device may also be used to deliver other types of drugs that have been shown to be difficult to delivery orally. For example, the transdermal delivery device may be used to deliver vitamin B12 directly into the bloodstream. This is particularly useful for patients who are vitamin B12 deficient or have a hard time with absorption of vitamin B12 who would otherwise have to give themselves intramuscular injections. Depending on the patient, the frequency of these types of injections may range from a weekly basis to even on a daily basis. The transdermal delivery device may also be used to delivery anti-inflammatories and other natural products including natural products that to this date have been difficult to deliver into the body such as curcumin. Also, the transdermal delivery device is effective for use in targeted delivery of drugs within specific areas of the body.

It is contemplated that certain state or federal requirements may require that indicia or labeling be present on the transdermal drug delivery device. Accordingly, the transdermal drug delivery device of the present disclosure allows for indicia to be adhered or alternatively, printed directly within the polymeric matrix. In particular, indicia may be adhered or printed on the non-delivering side of the polymeric matrix allowing for the indicia to remain in the polymeric matrix and not pressed onto the skin. The method for printing such indicia on the transdermal drug delivery device includes printing indicia on the non-delivering side of the polymeric matrix as a non-delivering agent and allowing the indicia to dry so that it does not pass through the polymeric matrix into the body.

### Example—Liposomal Emulsion Preparation

Materials

Consumables
Cold, distilled water (5.5 g)
Hyaluronic acid (0.050 g)
MCT oil (30 g)
Soy lecithin (1.5 g)
Food grade grain ethanol (3.5 g)
Serological pipettes (10 mL, 25 mL)

Equipment
150 mL Erlenmeyer flask (2×)
2 L bath sonicator
Analytical balance
Pipette pump

Procedure—Preparation of Emulsion
1. Weigh 5.5 g of cold water into a clean dry flask (Flask 1). Add 0.05 g hyaluronic acid and mix thoroughly with sonication. The solution should form a clear gel. Set Flask 1 aside.
2. Weigh 30 g MCT oil into a clean dry flask (Flask 2). Prepare a 5% w/w solution of soy lecithin in the MCT oil (1.5 g lecithin into 30 g oil). Mix thoroughly with sonication.
3. Slowly add 20.0 g of the 5% lecithin solution of Flask 2 into Flask 1 with through mixing and sonication (2 g at a time). Add approx. 3.5 g ethanol and continue mixing. Lastly, dissolve 5.0 g CBD distillate into flask 1 and sonicate. The resulting emulsion will appear a milky white and should not leave any solid particulate on the side of the flask upon swirling.

Patch Production
1. A suitable medical grade contact adhesive is selected for the application such as Henkel Duro-Tak 87-9301. The ratio of adhesive to emulsion will depend on several factors including patch dosage, size, and thickness in addition to other chemical/physical properties of both the drug and adhesive selected.
2. Once the emulsion and adhesive are mixed, they are laminated onto a release paper with an adjustable (thickness and width) pinch roller type device. While increasing thickness increases dosage in an otherwise equivalent form factor, it comes at the expense of delayed onset. The width of lamination and the length pulled determines the yield of patches produced and is optimized to the width/layout of the final die cutter.
3. After the emulsion-adhesive mixture has been laminated, a drying step is utilized to remove volatile compounds (solvents, water . . . etc.). A short room temperature bake (5-15 minutes) in a well ventilated, climate-controlled area is followed by a by a 2^{nd} short bake in an oven (either inline, much akin to a sub oven at a sandwich shop, in an automated scenario or separate if performed by hand) at modest temperature (<110° C.). Once dry, the outward facing side of the final matrix can be printed on by inkjet. Next, release paper is laminated onto the exposed side of the matrix using a similar pinch roller device as before. The final sheet (the patch matrix sandwiched between layers of release paper, which itself is inkjet printable as well) is then fed into a die cutter (either automated or cranked by hand). The now loose patches are then packaged for sale.

Solubility

In considering the advantage of dissolving cannabidiol (CBD) in the polymeric-emulsion set forth herein, consideration is first given to the solubility of CBD in pure water. Pure water has an accepted solubility value with respect to CBD of 0.7 μg per mL of water. (E. Samara and M. Bialer, Drug Metab. Dispos., 16 (6) 875-879 (1988). This provides a maximum weight/weight percent of approximately 0.07%.

In certain embodiments of the present teaching, hyaluronic acid may be present in the polymeric-emulsion disclosed herein. Such embodiments also include the presence of water. In one embodiment including hyaluronic acid, MCT (medium-chain triglycerides) oil was selected as a hydrophobic component as it can create stable solutions at a concentration of 400 mg/mL CBD or 40% by weight CBD. In the present case, this value was slightly exceeded in testing the liposomal polymeric emulsion set forth herein in combination with hyaluronic acid, water and MCT as the hydrophobic component. Here, the combination described above allows for the ability to obtain a solution having a concentration of 450 mg/ML CBD or 45% by weight CBD to be obtained. That said, the polymeric liposomal emulsion set forth herein retains the ability to dissolve water soluble compounds even when incorporating oil-soluble compounds at a relatively high percentage or amount. When compared to water, a greater than 500-fold increase in CBD solubility is observed and when compared to base MCT oil, a 1.125-fold increase in CBD solubility is observed.

Delivery Rate

Liposomal systems in general have been demonstrated to deliver compounds of comparable size and hydrophobicity at a 30-fold rate increase when compared to naked drugs. (Kim T., Murdande S., Gruber A., Kim S. Sustained-release morphine for epidural analgesia in rats. J. Am. Soc. Anesthesiol. 1996; 85:331-338. Doi:10.1097/00000542-199608000-00015). The polymeric liposomal emulsion of the present disclosure is expected to exhibit a comparable increase in the delivery rate.

Bioavailability

Cannabidiol (CBD) and other cannabinoids are known to have a notoriously low naked bioavailability, often reported at 6%. (Perucca E, Bialer M (Jun. 5, 2020). “Critical Aspects Affecting Cannabidiol Oral Bioavailability and Metabolic Elimination, and Related Clinical Implications”. CNS Drugs. 34 (8): 795-800. doi:10.1007/s40263-020-00741-5). This leaves room for significant improvement. Liposomal systems in general have been demonstrated a 2.5 fold increase in bioavailability. (Yun-Kyoung Song, Seo Yeon Hyun, Hyung-Tae Kim, Chong-Kook Kim & Jung-Mi Oh (2011) Transdermal delivery of low molecular weight heparin loaded in flexible liposomes with bioavailability enhancement: comparison with ethosomes, Journal of Microencapsulation, 28:3, 151-158, DOI: 10.3109/02652048.2010.507880). The polymeric liposomal emulsion of the present disclosure is expected to exhibit a comparable increase in bioavailability.

Concentration

The polymeric liposomal emulsion disclosed herein, in a “when prepared” condition (i.e., as prepared for and incorporated into a transdermal drug delivery device), has a maximum concentration of 26.36% CBD in the polymeric liposomal emulsion at 14.16% emulsion in adhesive for a fully transparent patch at a wet thickness of 0.015 inches. Trials run up to 30% broad spectrum CBD extract at 14.16 liposomal emulsion have achieved a transparent patch. Polymeric liposomal emulsions having up to 45% broad spectrum CBD by mass in the emulsion may be obtainable if a pure CBD distillate is used.

In one example, 39.45 grams of emulsion is prepared from 10.4 grams of broad spectrum CBD extract, 19 grams medium chain triglyceride oil, 5.5 grams of water, 3.5 grams of ethanol, 1 gram of lecithin, and 0.05 grams of hyaluronic acid. This is added to 239.15 grams of adhesive and used to produce patches measuring 1.75 inches by 1.75 inches that contain 12 mg of CBD.

In another example, 39.45 grams of emulsion is prepared from 5.2 grams of broad spectrum CBD extract, 5.2 grams of CBG distillate, 19 grams medium chain triglyceride oil, 5.5 grams of water, 3.5 grams of ethanol, 1 gram of lecithin, and 0.05 grams of hyaluronic acid. This is added to 239.15 grams of adhesive and used to produce patches measuring 1.75 inches by 1.75 inches that contain 12 mg of cannabinoids in a 1:1 ratio of CBD to CBG (cannibigerol).

In another example, 39.45 grams of emulsion is prepared from 1.3 grams of broad spectrum CBD extract, 9.1 grams of CBG distillate, 19 grams medium chain triglyceride oil, 5.5 grams of water, 3.5 grams of ethanol, 1 gram of lecithin, and 0.05 grams of hyaluronic acid. This is added to 239.15 grams of adhesive and used to produce patches measuring 1.75 inches by 1.75 inches that contain 12 mg of cannabinoids in a 7:1 ratio of CBG (cannabigerol) to CBD.

The values described above may be optimized for maximal deliverable dosage at complete transparency. A thinner thickness may incorporate a larger percentage of emulsion but may have a smaller complete dosage. Likewise, a thicker patch may support a greater total dose at a lower percentage of emulsion but may not achieve full transparency or full delivery rate as the medication would need to travel a greater distance to reach the skin. Tolerance for each of the components of the composition in all of the examples set forth above is +/−0.1 mg.

Additional Applications

The flexibility of the emulsion formulation described above, including the nanoemulsion formulation, provides a great advantage providing a universal formulation that can be used in numerous applications and product types. This is due in part to the nature of the particles formed in the final emulsion which allow for the ratio of hydrophilic to hydrophobic solvents across the full spectrum ranging from primarily oil to primarily water and is amendable to spray drying for extending shelf life and other ease of use benefits. This enables the base emulsion to be used in nearly every product type imaginable. The emulsion is capable of improving the efficacy of a variety of transdermal products including but not limited to creams, balms, moisturizers, lotions, oils, patches, and adhesives that may be used to adhere tapes, bandages, support wraps, and other medical devices. It is also capable of improving the efficacy of other products including but not limited to soaps, bath bombs, perfumes, colognes, deodorants and edible products (e.g., cookies, gummies, chews, gum, bubble gum, bars, suckers, hard candies, chocolates, cakes, muffins, scones, etc.), tinctures, sublinguals, capsules, tablets, baking ingredients (e.g., spray dried powder enriched flours, sugar, artificial sweeteners, flavored emulsions). The nanoemulsion formulation may also be integrated into solvent systems that can be used to infuse a physical drug carrier, an example of which may include a toothpick or any other porous material.

With respect to edible products, the emulsion and nanoemulsion formulation may also incorporate vitamin B12 and vitamin B12 natural carrier proteins discussed above. It is to be understood, however, that variations in the consumption method of the various products even if in the same delivery mode may produce a variety of different results. For example, chewing gum remains in the mouth for an extended period of time and stimulates significant saliva production and as such is capable of recruiting endogenous haptocorrin and achieving high levels of bioavailability. In comparison, food type products such as cookies or gummies, remain in the mouth for a comparatively brief period of time and have less opportunity to interact with endogenous haptocorrin and as such, demonstrates much lower delivery throughput. However, recombinant protein technology may be used to incorporate supplemental haptocorrin and/or intrinsic factor into the edibles to bridge this gap. Additionally, for transdermal applications, supplemental transcobalamin and/or haptocorrin may improve efficiency in comparison to relying upon the recruitment of endogenous transcobalamin in the bloodstream. Also, for targeted delivery to the liver or kidneys, supplemental haptocorrin is expected to have a major impact, as it only represents a small fraction of the endogenous production in the bloodstream.

While the present disclosure has been described with reference to various exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the bet mode contemplated for carrying out the invention, but that the invention will include all embodiments falling within the scope of the appended claims. Further, the “invention” as that term is used in this document is what is claimed in the claims of this document. The right to claim elements and/or sub-combinations that are disclosed herein as other inventions in other patent documents is hereby unconditionally reserved.

Having thus described the above subject matter, it is now claimed:

What is claimed is:

1. A transdermal drug delivery device comprising a single-layered polymeric matrix comprising a liposomal emulsion having a core drug reservoir and an adhesive.

2. The transdermal drug delivery device of claim 1, wherein the liposomal emulsion is a nanoparticle liposomal emulsion.

3. The transdermal drug delivery device of claim 2, wherein the nanoparticle liposomal emulsion holds oil soluble compounds within its core.

4. The transdermal drug delivery device of claim 3, wherein the oil soluble compounds within the core comprises at least one of cannabidiol (CBD), cannibigerol (CBG) and tetrahydrocannabinol (THC).

5. The transdermal drug delivery device of claim 4, wherein the polymeric matrix comprises an acrylates copolymer.

6. The transdermal drug delivery device of claim 5, wherein the adhesive comprises one or more of the following solvents: ethyl acetate, heptanes, n-heptane, hexane, methanol, ethanol, isopropanol, 2,4-pentanedione, toluene, and xylene.

7. The transdermal drug delivery device of claim 6, wherein the adhesive comprises ethyl acetate.

8. The transdermal drug delivery device of claim 1, wherein the transdermal drug delivery device has a transparency of greater than 80%.

9. The transdermal drug delivery device of claim 3, wherein the emulsion further comprises hyaluronic acid.

10. The transdermal drug delivery device of claim 3, wherein the polymeric matrix further comprises vitamin B12 and natural carrier proteins outside of the liposomes within the polymeric matrix.

11. The transdermal drug delivery device of claim 3, wherein the onset time is from about 5 to about 15 minutes.

12. The transdermal drug delivery device of claim 3, wherein about 80% of the drug is administered in about 30 to about 60 minutes.

13. The transdermal drug delivery device of claim 3, wherein the core comprises vitamin B12.

14. The transdermal drug delivery device of claim 1, wherein indicia is printed on a non-delivering side within the polymeric matrix.

15. The transdermal drug delivery device of claim 4, wherein a concentration of CBD in the liposomal emulsion of 26.36% and wherein a concentration of emulsion in adhesive of 14.16% achieves a fully transparent patch.

16. The transdermal drug delivery device of claim 4 comprising a patch, further comprising one of the following formulations:
iii) 39.35 to 39.55 grams of emulsion and 239.05 to 239.25 grams of adhesive for producing patches measuring about 1.75 inches by about 1.75 inches that contains about 12 mg of CBD, wherein the emulsion is prepared from 10.3 to 10.5 grams of broad spectrum CBD extract, 18.9 to 19.1 grams medium chain triglyceride oil, 5.4 to 5.6 grams of water, 3.4 to 3.6 grams of ethanol, 0.9 to 1.1 grams of lecithin, and 0 to 0.15 grams of hyaluronic acid;
iv) 39.35 to 39.55 grams of emulsion and 239.05 to 239.25 grams of adhesive for producing patches measuring about 1.75 inches by about 1.75 inches that contain about 12 mg of cannabinoids in a 1:1 ratio of CBD to CBG, wherein the emulsion is prepared from 5.1 to 5.3 grams of broad spectrum CBD extract, 5.1 to 5.3 grams of CBG distillate, 18.9 to 19.1 grams medium chain triglyceride oil, 5.4 to 5.6 grams of water, 3.4 to 3.6 grams of ethanol, 0.9 to 1.1 grams of lecithin, and 0 to 0.15 grams of hyaluronic acid;
iv.) 39.35 to 39.55 grams of emulsion and 239.05 to 239.25 grams of adhesive for producing patches measuring about 1.75 inches by about 1.75 inches that contain about 12 mg of cannabinoids in a 7:1 ratio of CBG to CBD, wherein the emulsion is prepared from 1.2 to 1.4 grams of broad spectrum CBD extract, 9.0 to 9.2 grams of CBG distillate, 18.9 to 19.1 grams medium chain triglyceride oil, 5.4 to 5.6 grams of water, 3.4 to 3.6 grams of ethanol, 0.9 to 1.1 grams of lecithin, and 0 to 0.15 grams of hyaluronic acid.

17. A gum comprising a polymeric matrix comprising a liposomal emulsion having a core drug reservoir and an adhesive.

18. The gum of claim 17, wherein the liposomal emulsion is a nanoparticle liposomal emulsion.

19. The gum of claim 18, wherein the nanoparticle liposomal emulsion holds oil soluble compounds within its core.

20. The gum of claim 19, wherein the oil soluble compounds within the core comprise at least one of cannabidiol (CBD) and tetrahydrocannabinol (THC).
